(19) United States
(12) Patent Application Publication
(10) Pub. No.: US 2006/0223067 A1
(43) Pub. Date: Oct. 5, 2006

(54) Mutant DNA polymerases and methods of use

(76) Inventors: Paolo Vatta, San Mateo, CA (US); John W. Brandis, Austin, TX (US); Elena V. Bolchakova, Union City, CA (US); Sandra L. Spurgeon, San Mateo, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/095,042

(22) Filed: Mar. 31, 2005

(51) Int. Cl. C12Q 1/68; C07H 21/04; C12P 21/06; C12N 9/22; C12N 15/74; C12N 1/21

(52) U.S. Cl. 435/6; 435/69.1; 435/199; 435/252.3; 435/471; 536/23.2

(57) ABSTRACT

The present invention provides mutant DNA polymerases, polynucleotides encoding the polymerases, cassettes and vectors including such polynucleotides, and cells containing the polymerases, polynucleotides, cassettes, and/or vectors of the invention. The present invention also provides methods for synthesizing polynucleotides and kits including a DNA polymerase of the invention.

### FIELD OF THE INVENTION

The invention is generally related to mutant DNA polymerases.

### BACKGROUND OF THE INVENTION

DNA polymerases are enzymes that synthesize DNA molecules using a template DNA strand and a complementary synthesis primer annealed to a portion of the template. A detailed description of DNA polymerases and their enzymological characterization can be found in Kornberg (1989).

The amino acid sequences of many DNA polymerases have been determined, and sequence comparisons between different DNA polymerases have identified many regions of homology between the different enzymes. Studies of the tertiary structures of DNA polymerases and amino acid sequence comparisons have revealed numerous structural similarities between diverse DNA polymerases. In general, DNA polymerases have a large cleft that is thought to accommodate the binding of duplex DNA. This cleft is formed by two sets of helices, the first set is referred to as the “fingers” region and the second set of helices is referred to as the “thumb” region. The bottom of the cleft is formed by anti-parallel beta sheets and is referred to as the “palm” region. Reviews of DNA polymerase structure can be found in Joyce and Steitz (1994). Computer readable data files describing the three-dimensional structure of some DNA polymerases have been publicly disseminated.

DNA polymerases have a variety of uses in molecular biology techniques suitable for both research and clinical applications. Foremost among these techniques are DNA sequencing and polynucleotide amplification techniques such as the polymerase chain reaction (PCR).

However, while widely used, available DNA polymerases can display any number of attributes that can decrease the enzyme's efficiency for synthesizing DNA, including: the polymerase may not efficiently read through all regions of the template; the polymerase may have decreased efficiency at higher salt concentrations; the polymerase may display 5′-3′ nuclease activity; and/or the polymerase may discriminate against the efficient incorporation of fluorescently labeled nucleotides into the resulting DNA strand.

Accordingly, there is a need for DNA polymerases having increased efficiency for synthesizing DNA molecules from, e.g., fluorescently labeled nucleotides.

### SUMMARY OF CERTAIN EMBODIMENTS OF THE INVENTION

Provided herein are mutant polymerases useful, e.g., for sequencing DNA. In some embodiments, the mutations of a mutant polymerase (1) decrease 5′-3′ nuclease activity; (2) allow for more efficient incorporation of fluorescently labeled nucleotides into the resulting DNA strand; (3) enhance the processivity of the polymerase; and/or (4) improve the ability of the polymerase to read through templates, e.g., with secondary structure.

Accordingly, certain embodiments of the present invention provide mutant DNA polymerase including an Asn residue at amino acid 543 and a 5′-3′ exonuclease activity reducing mutation, wherein the positions of amino acids of the mutant DNA polymerase are defined with respect to Taq DNA polymerase. In certain embodiments, the 5′-3′ exonuclease activity reducing mutation is an N-terminal deletion. In certain embodiments, the 5′-3′ exonuclease activity reducing mutation is an Asp residue at amino acid 46. The polymerase may also include a Tyr residue at amino acid 667.

The invention also provides in certain embodiments polynucleotides encoding the polymerases of the invention, expression cassettes and vectors including such polynucleotides, and cells containing such polymerases and polynucleotides.

Also provided are methods for synthesizing polynucleotides in a reaction, including contacting at least one polymerase of the invention with a primed template and nucleotides, e.g., fluorescently labeled nucleotides, under conditions effective to synthesize polynucleotides. The present invention in certain embodiments also provides kits containing packaging material and at least one polymerase of the invention.

Also provided are methods for sequencing polynucleotides, e.g., sequencing a DNA sequence, using a polymerase of the invention.

### BRIEF DESCRIPTION OF THE FIGURES

FIG. 1 depicts kinetic steps in the forward polymerization pathway for Taq G46D, F667Y.

FIG. 2 depicts the principle kinetic steps for processive polymerization under conditions where only dATP, dCTP, and dTTP nucleotides are included in the reaction mixture.

FIG. 3 depicts processive polymerization by Taq G46D, F667Y on 36/45-mer DNA.

FIG. 4 depicts processive polymerization by Taq G46D, F667Y on 36/45-mer DNA.

FIG. 5 depicts the polymerization and dissociation rates for Taq G46D, F667Y.

FIG. 6 depicts processive polymerization by triple mutant Taq G46D, S543N, F667Y on 36/45-mer DNA.

FIG. 7 depicts a processive polymerization pathway for Taq G46D, F667Y, S543N.

### DETAILED DESCRIPTION OF THE INVENTION

Described herein are polymerases that combine mutations to produce an enhanced polymerase useful, e.g., for sequencing DNA. In some embodiments, these mutations (1) decrease 5′-3′ nuclease activity; (2) allow for more efficient incorporation of fluorescently labeled nucleotides into the resulting DNA strand; (3) enhance the processivity of the polymerase; and/or (4) improve the ability of the polymerase to read through regions in templates that can cause sequencing failures with other polymerases.

Accordingly, certain embodiments of the present invention provide mutant DNA polymerase including an Asn residue at amino acid 543 and a 5′-3′ exonuclease activity reducing mutation, wherein the positions of amino acids of the mutant DNA polymerase are defined with respect to Taq DNA polymerase. In certain embodiments, the 5′-3′ exonuclease activity reducing mutation is an N-terminal deletion. In certain embodiments, the 5′-3′ exonuclease activity reducing mutation is an Asp residue at amino acid 46. The polymerase may also include a Tyr residue at amino acid 667. The DNA polymerase may be a thermostable DNA polymerase. The DNA polymerase may be a mutated Taq DNA polymerase. The DNA polymerase may be a thermostable Taq DNA polymerase. In certain embodiments, the DNA polymerase may include SEQ ID NO:3 or SEQ ID NO:5.

The present invention also provides polynucleotides encoding the polymerases of the invention, such as SEQ ID NO:4 and SEQ ID NO:6, and cassettes and vectors including such polynucleotides. The polynucleotide may be operably linked to a promoter. Also provided are cells containing the polymerases, polynucleotides, cassettes, and/or vectors of the invention.

A wild type polymerase from Thermus aquaticus is SEQ ID NO:1. A nucleotide sequence encoding such a wild type polymerase is SEQ ID NO:2. (see accession number J04636)

(SEQ ID NO: 1)

MRGMLPLFEPKGRVLLVDGHHLAYRTFHALKGLTTSRGEPVQAVYGFAKS
LLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLS
DRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWP
PPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTE
EAGERAALSERLFANLWGRLEGEERLLWLYREVERPLSAVLAHMEATGVR
LDVAYLRALSLEVAEEIARLEAEVFRLAGHPFNLNSRDQLERVLFDELGL
PAIGKTEKTGKRSTSAAVLEALREAHPIVEKILQYRELTKLKSTYIDPLP
DLIHPRTGRLHTRFNQTATATGRLSSSDPNLQNIPVRTPLGQRIRRAFIA
EEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFN
MPVQGTAADLMKLAMVKLFPRLEEMGARMLLQVHDELVLEAPKERAEAVA
RLAKEVMEGVYPLAVPLEVEVGIGEDWLSAKE

In the sequence below, the start codon (atg) at position 121 is underlined. Also underlined are codons that may be mutated in some embodiments of the invention to produce a polymerase of the invention.

(SEQ ID NO: 2)

| | |
|---|---|
| 1 | aagctcagat ctacctgcct gagggcgtcc ggttccagct |
| | ggcccttccc gagggggaga |
| 61 | gggaggcgtt tctaaaagcc cttcaggacg ctacccgggg |
| | gcgggtggtg gaagggtaac |
| 121 | atgaggggga tgctgcccct ctttgagccc aagggccggg |
| | tcctcctggt ggacggccac |
| 181 | cacctggcct accgcacctt ccacgccctg aagggcctca |
| | ccaccagccg gggggagccg |
| 241 | gtgcaggcgg tctacggctt cgccaagagc ctcctcaagg |
| | ccctcaagga ggacggggac |
| 301 | gcggtgatcg tggtctttga cgccaaggcc ccctccttcc |
| | gccacgaggc ctacgggggg |
| 361 | tacaaggcgg gccgggcccc cacgccggag gactttcccc |
| | ggcaactcgc cctcatcaag |
| 421 | gagctggtgg acctcctggg gctggcgcgc ctcgaggtcc |
| | cgggctacga ggcggacgac |
| 481 | gtcctggcca gcctggccaa gaaggcggaa aaggagggct |
| | acgaggtccg catcctcacc |
| 541 | gccgacaaag acctttacca gctcctttcc gaccgcatcc |
| | acgtcctcca ccccgagggg |
| 601 | tacctcatca ccccggcctg gctttgggaa aagtacggcc |
| | tgaggcccga ccagtgggcc |
| 661 | gactaccggg ccctgaccgg ggacgagtcc gacaaccttc |
| | ccggggtcaa gggcatcggg |
| 721 | gagaagacgg cgaggaagct tctggaggag tgggggagcc |
| | tggaagccct cctcaagaac |
| 781 | ctggaccggc tgaagcccgc catccgggag aagatcctgg |
| | cccacatgga cgatctgaag |
| 841 | ctctcctggg acctggccaa ggtgcgcacc gacctgcccc |
| | tggaggtgga cttcgccaaa |
| 901 | aggcgggagc ccgaccggga gaggcttagg gcctttctgg |
| | agaggcttga gtttggcagc |
| 961 | ctcctccacg agttcggcct tctggaaagc cccaaggccc |
| | tggaggaggc cccctggccc |
| 1021 | ccgccggaag gggccttcgt gggctttgtg ctttcccgca |
| | aggagcccat gtgggccgat |
| 1081 | cttctggccc tggccgccgc cagggggggc cgggtccacc |
| | gggcccccga gccttataaa |
| 1141 | gccctcaggg acctgaagga ggcgcggggg cttctcgcca |
| | aagacctgag cgttctggcc |
| 1201 | ctgagggaag gccttggcct cccgcccggc gacgacccca |
| | tgctcctcgc ctacctcctg |
| 1261 | gacccttcca acaccacccc cgagggggtg gcccggcgct |
| | acggcgggga gtggacggag |
| 1321 | gaggcggggg agcgggccgc cctttccgag aggctcttcg |
| | ccaacctgtg ggggaggctt |
| 1381 | gagggggagg agaggctcct ttggctttac cgggaggtgg |
| | agaggcccct ttccgctgtc |
| 1441 | ctggcccaca tggaggccac gggggtgcgc ctggacgtgg |
| | cctatctcag ggccttgtcc |
| 1501 | ctggaggtgg ccgaggagat cgcccgcctc gaggccgagg |
| | tcttccgcct ggccggccac |
| 1561 | cccttcaacc tcaactcccg ggaccagctg gaaagggtcc |
| | tctttgacga gctagggctt |
| 1621 | cccgccatcg gcaagacgga gaagaccggc aagcgctcca |
| | ccagcgccgc cgtcctggag |
| 1681 | gccctccgcg aggcccaccc catcgtggag aagatcctgc |
| | agtaccggga gctcaccaag |
| 1741 | ctgaagagca cctacattga ccccttgccg gacctcatcc |
| | accccaggac gggccgcctc |
| 1801 | cacacccgct tcaaccagac ggccacggcc acgggcaggc |
| | taagtagctc cgatcccaac |
| 1861 | ctccagaaca tccccgtccg caccccgctt gggcagagga |
| | tccgccgggc cttcatcgcc |
| 1921 | gaggaggggt ggctattggt ggccctggac tatagccaga |
| | tagagctcag ggtgctggcc |
| 1981 | cacctctccg gcgacgagaa cctgatccgg gtcttccagg |
| | aggggcggga catccacacg |
| 2041 | gagaccgcca gctggatgtt cggcgtcccc cgggaggccg |
| | tggaccccct gatgcgccgg |
| 2101 | gcggccaaga ccatcaac tt cggggtcctc tacggcatgt |
| | cggcccaccg cctctcccag |
| 2161 | gagctagcca tcccttacga ggaggcccag gccttcattg |
| | agcgctactt tcagagcttc |
| 2221 | cccaaggtgc gggcctggat tgagaagacc ctggaggagg |
| | gcaggaggcg ggggtacgtg |
| 2281 | gagaccctct tcggccgccg ccgctacgtg ccagacctag |
| | aggcccgggt gaagagcgtg |
| 2341 | cgggaggcgg ccgagcgcat ggccttcaac atgcccgtcc |
| | agggcaccgc cgccgacctc |
| 2401 | atgaagctgg ctatggtgaa gctcttcccc aggctggagg |
| | aaatgggggc caggatgctc |
| 2461 | cttcaggtcc acgacgagct ggtcctcgag gccccaaaag |
| | agagggcgga ggccgtggcc |
| 2521 | cggctggcca aggaggtcat ggagggggtg tatcccctgg |
| | ccgtgcccct ggaggtggag |
| 2581 | gtggggatag gggaggactg gctctccgcc aaggagtgat |
| | accacc |

A mutant DNA polymerase of the invention (G46D, S543N, F667Y; SEQ ID NO:3) is provided below. A nucleotide sequence encoding such a polymerase is SEQ ID NO:4. The start codon atg is at position 121 and is underlined below. Also underlined are mutated amino acids and codons.

(SEQ ID NO: 3)

MRGMLPLFEPKGRVLLVDGHHLAYRTFHALKGLTTSRGEPVQAVYDFAKS
LLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLS
DRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWP
PPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTE
EAGERAALSERLFANLWGRLEGEERLLWLYREVERPLSAVLAHMEATGVR
LDVAYLRALSLEVAEEIARLEAEVFRLAGHPFNLNSRDQLERVLFDELGL
PAIGKTEKTGKRSTSAAVLEALREAHPIVEKILQYRELTKLKNTYIDPLP
DLIHPRTGRLHTRFNQTATATGRLSSSDPNLQNIPVRTPLGQRIRRAFIA
EEGWLLVALDYSQIELRVLAHLSGDENLIRVFQEGRDIHTETASWMFGVP
REAVDPLMRRAAKTINYGVLYGMSAHRLSQELA
IPYEEAQAFIERYFQSFPKVRAWIEKTLEEGRRRGYVETLFGRRRYVPDL
EARVKSVREAAERMAFNMPVQGTAADLMKLAMVKLFPRLEEMGARMLLQV
HDELVLEAPKERAEAVARLAKEVMEGVYPLAVPLEVEVGIGEDWLSAKE

(SEQ ID NO: 4)

| | |
|---|---|
| 1 | aagctcagat ctacctgcct gagggcgtcc ggttccagct |
| | ggcccttccc gagggggaga |
| 61 | gggaggcgtt tctaaaagcc cttcaggacg ctacccgggg |
| | gcgggtggtg gaagggtaac |
| 121 | atgaggggga tgctgcccct ctttgagccc aagggccggg |
| | tcctcctggt ggacggccac |
| 181 | cacctggcct accgcacctt ccacgccctg aagggcctca |
| | ccaccagccg gggggagccg |
| 241 | gtgcaggcgg tctacgactt cgccaagagc ctcctcaagg |
| | ccctcaagga ggacggggac |
| 301 | gcggtgatcg tggtctttga cgccaaggcc ccctccttcc |
| | gccacgaggc ctacgggggg |
| 361 | tacaaggcgg gccgggcccc cacgccggag gactttcccc |
| | ggcaactcgc cctcatcaag |
| 421 | gagctggtgg acctcctggg gctggcgcgc ctcgaggtcc |
| | cgggctacga ggcggacgac |
| 481 | gtcctggcca gcctggccaa gaaggcggaa aaggagggct |
| | acgaggtccg catcctcacc |
| 541 | gccgacaaag acctttacca gctcctttcc gaccgcatcc |
| | acgtcctcca ccccgagggg |
| 601 | tacctcatca ccccggcctg gctttgggaa aagtacggcc |
| | tgaggcccga ccagtgggcc |
| 661 | gactaccggg ccctgaccgg ggacgagtcc gacaaccttc |
| | ccggggtcaa gggcatcggg |
| 721 | gagaagacgg cgaggaagct tctggaggag tgggggagcc |
| | tggaagccct cctcaagaac |
| 781 | ctggaccggc tgaagcccgc catccgggag aagatcctgg |
| | cccacatgga cgatctgaag |
| 841 | ctctcctggg acctggccaa ggtgcgcacc gacctgcccc |
| | tggaggtgga cttcgccaaa |
| 901 | aggcgggagc ccgaccggga gaggcttagg gcctttctgg |
| | agaggcttga gtttggcagc |
| 961 | ctcctccacg agttcggcct tctggaaagc cccaaggccc |
| | tggaggaggc cccctggccc |
| 1021 | ccgccggaag gggccttcgt gggctttgtg ctttcccgca |
| | aggagcccat gtgggccgat |
| 1081 | cttctggccc tggccgccgc cagggggggc cgggtccacc |
| | gggcccccga gccttataaa |
| 1141 | gccctcaggg acctgaagga ggcgcggggg cttctcgcca |
| | aagacctgag cgttctggcc |
| 1201 | ctgagggaag gccttggcct cccgcccggc gacgacccca |
| | tgctcctcgc ctacctcctg |
| 1261 | gacccttcca acaccacccc cgagggggtg gcccggcgct |
| | acggcgggga gtggacggag |
| 1321 | gaggcggggg agcgggccgc cctttccgag aggctcttcg |
| | ccaacctgtg ggggaggctt |
| 1381 | gagggggagg agaggctcct ttggctttac cgggaggtgg |
| | agaggcccct ttccgctgtc |
| 1441 | ctggcccaca tggaggccac gggggtgcgc ctggacgtgg |
| | cctatctcag ggccttgtcc |
| 1501 | ctggaggtgg ccgaggagat cgcccgcctc gaggccgagg |
| | tcttccgcct ggccggccac |
| 1561 | cccttcaacc tcaactcccg ggaccagctg gaaagggtcc |
| | tctttgacga gctagggctt |
| 1621 | cccgccatcg gcaagacgga gaagaccggc aagcgctcca |
| | ccagcgccgc cgtcctggag |
| 1681 | gccctccgcg aggcccaccc catcgtggag aagatcctgc |
| | agtaccggga gctcaccaag |
| 1741 | ctgaagaata cctacattga ccccttgccg gacctcatcc |
| | accccaggac gggccgcctc |
| 1801 | cacacccgct tcaaccagac ggccacggcc acgggcaggc |
| | taagtagctc cgatcccaac |
| 1861 | ctccagaaca tccccgtccg caccccgctt gggcagagga |
| | tccgccgggc cttcatcgcc |
| 1921 | gaggaggggt ggctattggt ggccctggac tatagccaga |
| | tagagctcag ggtgctggcc |
| 1981 | cacctctccg gcgacgagaa cctgatccgg gtcttccagg |
| | aggggcggga catccacacg |
| 2041 | gagaccgcca gctggatgtt cggcgtcccc cgggaggccg |
| | tggaccccct gatgcgccgg |
| 2101 | gcggccaaga ccatcaac tac ggggtcctc tacggcatgt |
| | cggcccaccg cctctcccag |
| 2161 | gagctagcca tcccttacga ggaggcccag gccttcattg |
| | agcgctactt tcagagcttc |
| 2221 | cccaaggtgc gggcctggat tgagaagacc ctggaggagg |
| | gcaggaggcg ggggtacgtg |
| 2281 | gagaccctct tcggccgccg ccgctacgtg ccagacctag |
| | aggcccgggt gaagagcgtg |
| 2341 | cgggaggcgg ccgagcgcat ggccttcaac atgcccgtcc |
| | agggcaccgc cgccgacctc |
| 2401 | atgaagctgg ctatggtgaa gctcttcccc aggctggagg |
| | aaatgggggc caggatgctc |
| 2461 | cttcaggtcc acgacgagct ggtcctcgag gccccaaaag |
| | agagggcgga ggccgtggcc |
| 2521 | cggctggcca aggaggtcat ggagggggtg tatcccctgg |
| | ccgtgcccct ggaggtggag |
| 2581 | gtggggatag gggaggactg gctctccgcc aaggagtgat |
| | accacc |

A mutant DNA polymerase of the invention (G46D, S543N; SEQ ID NO:5) is provided below. A nucleotide sequence encoding such a polymerase is SEQ ID NO:6. The start codon atg is at position 121 and is underlined below. Also underlined are mutated amino acids and codons.

(SEQ ID NO: 5)

MRGMLPLFEPKGRVLLVDGHHLAYRTFHALKGLTTSRGEPVQAVYDFAKS
LLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLS
DRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWP
PPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTE
EAGERAALSERLFANLWGRLEGEERLLWLYREVERPLSAVLAHMEATGVR
LDVAYLRALSLEVAEEIARLEAEVFRLAGHPFNLNSRDQLERVLFDELGL
PAIGKTEKTGKRSTSAAVLEALREAHPIVEKILQYRELTKLKNTYIDPLP
DLIHPRTGRLHTRFNQTATATGRLSSSDPNLQNIPVRTPLGQRIRRAFIA
EEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFN
MPVQGTAADLMKLAMVKLFPRLEEMGARMLLQVHDELVLEAPKERAEAVA
RLAKEVMEGVYPLAVPLEVEVGIGEDWLSAKE

(SEQ ID NO: 6)

| | |
|---|---|
| 1 | aagctcagat ctacctgcct gagggcgtcc ggttccagct |
| | ggcccttccc gagggggaga |
| 61 | gggaggcgtt tctaaaagcc cttcaggacg ctacccgggg |
| | gcgggtggtg gaagggtaac |
| 121 | atgaggggga tgctgcccct ctttgagccc aagggccggg |
| | tcctcctggt ggacggccac |
| 181 | cacctggcct accgcacctt ccacgccctg aagggcctca |
| | ccaccagccg gggggagccg |
| 241 | gtgcaggcgg tctacgactt cgccaagagc ctcctcaagg |
| | ccctcaagga ggacggggac |
| 301 | gcggtgatcg tggtctttga cgccaaggcc ccctccttcc |
| | gccacgaggc ctacgggggg |
| 361 | tacaaggcgg gccgggcccc cacgccggag gactttcccc |
| | ggcaactcgc cctcatcaag |
| 421 | gagctggtgg acctcctggg gctggcgcgc ctcgaggtcc |
| | cgggctacga ggcggacgac |
| 481 | gtcctggcca gcctggccaa gaaggcggaa aaggagggct |
| | acgaggtccg catcctcacc |
| 541 | gccgacaaag acctttacca gctcctttcc gaccgcatcc |
| | acgtcctcca ccccgagggg |
| 601 | tacctcatca ccccggcctg gctttgggaa aagtacggcc |
| | tgaggcccga ccagtgggcc |
| 661 | gactaccggg ccctgaccgg ggacgagtcc gacaaccttc |
| | ccggggtcaa gggcatcggg |
| 721 | gagaagacgg cgaggaagct tctggaggag tgggggagcc |
| | tggaagccct cctcaagaac |
| 781 | ctggaccggc tgaagcccgc catccgggag aagatcctgg |
| | cccacatgga cgatctgaag |
| 841 | ctctcctggg acctggccaa ggtgcgcacc gacctgcccc |
| | tggaggtgga cttcgccaaa |
| 901 | aggcgggagc ccgaccggga gaggcttagg gcctttctgg |
| | agaggcttga gtttggcagc |
| 961 | ctcctccacg agttcggcct tctggaaagc cccaaggccc |
| | tggaggaggc cccctggccc |
| 1021 | ccgccggaag gggccttcgt gggctttgtg ctttcccgca |
| | aggagcccat gtgggccgat |
| 1081 | cttctggccc tggccgccgc cagggggggc cgggtccacc |
| | gggcccccga gccttataaa |
| 1141 | gccctcaggg acctgaagga ggcgcggggg cttctcgcca |
| | aagacctgag cgttctggcc |
| 1201 | ctgagggaag gccttggcct cccgcccggc gacgacccca |
| | tgctcctcgc ctacctcctg |
| 1261 | gacccttcca acaccacccc cgagggggtg gcccggcgct |
| | acggcgggga gtggacggag |
| 1321 | gaggcggggg agcgggccgc cctttccgag aggctcttcg |
| | ccaacctgtg ggggaggctt |
| 1381 | gagggggagg agaggctcct ttggctttac cgggaggtgg |
| | agaggcccct ttccgctgtc |
| 1441 | ctggcccaca tggaggccac gggggtgcgc ctggacgtgg |
| | cctatctcag ggccttgtcc |
| 1501 | ctggaggtgg ccgaggagat cgcccgcctc gaggccgagg |
| | tcttccgcct ggccggccac |
| 1561 | cccttcaacc tcaactcccg ggaccagctg gaaagggtcc |
| | tctttgacga gctagggctt |
| 1621 | cccgccatcg gcaagacgga gaagaccggc aagcgctcca |
| | ccagcgccgc cgtcctggag |
| 1681 | gccctccgcg aggcccaccc catcgtggag aagatcctgc |
| | agtaccggga gctcaccaag |
| 1741 | ctgaagaata cctacattga ccccttgccg gacctcatcc |
| | accccaggac gggccgcctc |
| 1801 | cacacccgct tcaaccagac ggccacggcc acgggcaggc |
| | taagtagctc cgatcccaac |
| 1861 | ctccagaaca tccccgtccg caccccgctt gggcagagga |
| | tccgccgggc cttcatcgcc |
| 1921 | gaggaggggt ggctattggt ggccctggac tatagccaga |
| | tagagctcag ggtgctggcc |
| 1981 | cacctctccg gcgacgagaa cctgatccgg gtcttccagg |
| | aggggcggga catccacacg |
| 2041 | gagaccgcca gctggatgtt cggcgtcccc cgggaggccg |
| | tggaccccct gatgcgccgg |
| 2101 | gcggccaaga ccatcaac ttc ggggtcctc tacggcatgt |
| | cggcccaccg cctctcccag |
| 2161 | gagctagcca tcccttacga ggaggcccag gccttcattg |
| | agcgctactt tcagagcttc |
| 2221 | cccaaggtgc gggcctggat tgagaagacc ctggaggagg |
| | gcaggaggcg ggggtacgtg |
| 2281 | gagaccctct tcggccgccg ccgctacgtg ccagacctag |
| | aggcccgggt gaagagcgtg |
| 2341 | cgggaggcgg ccgagcgcat ggccttcaac atgcccgtcc |
| | agggcaccgc cgccgacctc |
| 2401 | atgaagctgg ctatggtgaa gctcttcccc aggctggagg |
| | aaatgggggc caggatgctc |
| 2461 | cttcaggtcc acgacgagct ggtcctcgag gccccaaaag |
| | agagggcgga ggccgtggcc |
| 2521 | cggctggcca aggaggtcat ggagggggtg tatcccctgg |
| | ccgtgcccct ggaggtggag |
| 2581 | gtggggatag gggaggactg gctctccgcc aaggagtgat |
| | accacc |

Certain embodiments of the present invention also provide methods for synthesizing a polynucleotide in a reaction, including contacting at least one DNA polymerase of the invention with a primed template and nucleotides. The reaction may be, e.g., a chain termination sequencing reaction or a polymerase chain reaction. The nucleotides may include labeled nucleotides, e.g., fluorescently labeled nucleotides.

Certain embodiments of the present invention also provide kits including packaging material and a DNA polymerase of the invention. The kit may contain nucleotides, e.g., labeled nucleotides, e.g., fluorescently labeled nucleotides. The kits may also include unlabeled nucleotides. The kits may also include at least one primer.

Thus, a new polymerase has been developed that combines mutations to produce an enhanced polymerase useful, e.g., in DNA sequencing. These mutations may include: G46D, which reduces, e.g., eliminates, the 5′-3′ nuclease activity; F667Y, which allows more efficient incorporation of dideoxy nucleotides; and S543N, which enhances the processivity of the polymerase. S543N also improves the ability of the polymerase to read through regions in templates with secondary structure that would normally disrupt the sequencing ability of the polymerase. In addition, the S543N mutation enhances the salt tolerance of the polymerase.

The art worker may chose to substitute other mutations known to reduce or eliminate the 5′-3 exonuclease activity in Taq (e.g., D144A), e.g., based upon studies with other Pol I-type enzymes. (see Xu et al., 1997) Some methods for reducing the 5′-3 exonuclease activity can be found in U.S. Pat. Nos. 5,405,774, 5,455,170, 5466,591, and 5,795,762, e.g., by using an N-terminal deletion. Mutations at position 46 other than G46D may also be used to produce reduced 5′-3 exonuclease activity.

Thus, methods utilizing certain polymerases of the invention will demonstrate a reduction in failures in sequencing due to template secondary structure. Certain polymerases also have increased salt tolerance, which reduces sensitivity of the polymerase to salts, e.g., carried over from template preparations or from PCR reactions. Use of certain polymerases also reduces the number of false stops in dye primer reactions. The mutations in certain polymerases also improve the ability of polymerases of the invention to tolerate dITP and dUTP in the extending strand.

The polymerases of the invention could be used to make, e.g., dye terminator sequencing kits or dye-labeled primer kits. The polymerases of the invention could also be used in, e.g., direct PCR sequencing chemistry, e.g., in combination with a polymerase without the F667Y mutation. In some embodiments of the invention, the polymerases of the invention may be used, e.g., with dye-labeled primers and/or dye-labeled terminators, e.g., to perform simultaneous amplification and sequencing.

The S543N Mutation

The DNA polymerases from 7 different species of Thermus were cloned purified, and characterized. The sequence of the gene was obtained for the DNA polymerase from T. filiformis, T. scotoductus, T. oshimaii, T. antranikianii, T. brokianus, T. igniterrai and from 9 strains of T. thermophilus. All of the thermophilus strains were found to have N at the position corresponding to Taq 543. Surprisingly, none of the other genes had N at the corresponding position. Unexpectedly, testing of the polymerases produced from filiformis, scotoductus, oshimaii and 5 of the thermophilus strains indicated that the thermophilus strains all exhibited enhanced salt tolerance and an enhanced ability to read through regions of secondary structure compared to Taq and the other polymerases. Based on these findings, mutant Taq polymerases were produced that included the S543N mutation, both alone and in combination with other mutations such as G46D and/or F667Y.

For example, a mutant was made from Taq which combined G46D, F667Y and S543N in a single protein. This polymerase has enhanced processivity compared to Taq not having S543N. This mutant also behaves like the thermophilus strains in terms of its ability to read through templates having certain regions of secondary structure, and also has salt tolerance similar to the thermophilus strains. This polymerase performs well in both sequencing reactions and in PCR.

Thus, embodiments of the invention include the mutant polymerases and polynucleotide sequences encoding the mutant polymerases Polynucleotide sequences encoding the mutant polymerases of the invention may be used for the recombinant production of the mutant polymerases. Polynucleotide sequences encoding mutant polymerases may be produced by a variety of methods. One method of producing polynucleotide sequences encoding mutant polymerases is by using site-directed mutagenesis to introduce desired mutations into polynucleotides encoding the parent, wild-type polymerase.

Polynucleotides encoding the mutant polymerases of the invention may be used for the recombinant expression of the mutant polymerases. Generally, the recombinant expression of the mutant polymerase is effected by introducing a polynucleotide encoding a mutant polymerase into an expression vector adapted for use in particular type of host cell. Thus, another aspect of the invention is to provide vectors including a polynucleotide encoding a mutant polymerase of the invention, such that the polymerase encoding polynucleotide is functionally inserted into the vector. The invention also provide host cells that include the vectors of the invention. Host cells for recombinant expression may be prokaryotic or eukaryotic. Example of host cells include bacterial cells, yeast cells, cultured insect cell lines, and cultured mammalian cells lines. A wide range of vectors, e.g., expression vectors, are well known in the art, and the expression of polymerases in recombinant cell systems is a well-established technique.

The invention also provides kits for synthesizing polynucleotides, e.g., fluorescently labeled polynucleotides. The kits may be adapted for performing specific polynucleotide synthesis procedures such as DNA sequencing or PCR. Kits of certain embodiments of the invention include a mutant DNA polymerase of the invention. Kits preferably contain instructions on how to perform the procedures for which the kits are adapted. Optionally, the kits may further include at least one other reagent for performing the method the kit is adapted to perform. Examples of such additional reagents include labeled nucleotides, unlabeled nucleotides, buffers, cloning vectors, restriction endonucleases, sequencing primers, and amplification primers. The reagents include in the kits of the invention may be supplied in premeasured units so as to provide for greater precision and accuracy.

The following terms are used to describe the sequence relationships between two or more polynucleotides or polypeptides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a segment of or the entirety of a specified sequence.

(b) As used herein, “comparison window” makes reference to a contiguous and specified segment of a polynucleotide or polypeptide sequence, wherein the polynucleotide or polypeptide sequence in the comparison window may include additions or deletions (i.e., gaps) compared to the reference sequence (which does not include additions or deletions) for optimal alignment of the sequences. Generally, the comparison window is at least 5, 10 or 20 contiguous nucleotides or polypeptide in length, and optionally can be 30, 40, 50, 100, or longer. Those of skill in the art understand that to avoid a high similarity to a reference sequence due to inclusion of gaps in the polynucleotide or polypeptide sequence, a gap penalty can be introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, PNAS, 85:2444 (1988); the algorithm of Karlin and Altschul, PNAS, 87:2264 (1990), modified as in. Karlin and Altschul, PNAS, 90:5873 (1993).

Computer implementation of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program and GAP, BESTFIT, BLAST, FASTA, and TFASTA in the Wisconsin Genetics Software Package. Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information. This algorithm generally involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test polynucleotide sequence is considered similar to a reference sequence if the smallest sum probability in a comparison of the test polynucleotide sequence to the reference polynucleotide sequence is less than about 0.1, more preferably less than about 0.01, and most preferably less than about 0.001.

To obtain gapped alignments for comparison purposes, Gapped BLAST can be utilized as described in Altschul et al., Nucleic Acids Res. 25:3389 (1997). Alternatively, PSI-BLAST can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See http://www.ncbi.n1m.nih.gov. Alignments may also be performed manually by inspection.

For purposes of the present invention, comparison of sequences for determination of percent sequence identity to the sequences disclosed herein is preferably made using the BlastN program (version 1.4.7 or later) with its default parameters, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two polynucleotide or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide or polypeptide sequence in the comparison window may include additions or deletions (i.e., gaps) as compared to the reference sequence (which does not include additions or deletions) for optimal alignment of the two sequences. The percentage is calculated by determining the number of positions at which the identical polynucleotide base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a sequence includes a sequence that has at least about 51%, 52%, 53%, 54%, 55%, 56%, 57%, 58%, 59%, 60%, 61%, 62%, 63%, 64%, 65%, 66%, 67%, 68%, or 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

Another indication that sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe polynucleotide and a target polynucleotide and embraces minor mismatches that can be accommodated by reducing the stringency of the hybridization media to achieve the desired detection of the target polynucleotide sequence.

“Stringent hybridization conditions” and “stringent hybridization wash conditions” in the context of polynucleotide hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermnal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of polynucleotides is found in Tijssen, Laboratory Techniques in Biochemistry and Molecular Biology Hybridization with Nucleic Acid Probes, part I chapter 2 “Overview of principles of hybridization and the strategy of polynucleotide probe assays” Elsevier, New York (1993). Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Polynucleotides that do not hybridize to each other under stringent conditions are still substantially identical if the proteins that they encode are substantially identical. This occurs, e.g., when a copy of a polynucleotide is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodiurn citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Thus, certain embodiments of the present invention are directed to polynucleotide and polypeptide sequences that specifically hybridize to, or are substantially identical to the polypeptide sequences of the polymerases of the invention and the polynucleotide sequences that encode such polypeptide sequences. The activity of such polymerases may be determined using assays known to the art worker.

The polymerases of certain embodiments of the invention include polymerases with substitutions of at least one amino acid residue in the polypeptide. In some embodiments of the invention, amino acid substitutions falling within the scope of the invention include those that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

Substitution of like amino acids can also be made on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In such changes, the substitution of amino acids whose hydrophilicity values can be within ±2, within ±1, or within ±0.5.

In one embodiment of the invention, the polymerase has a conservative amino acid substitution, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitutions also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

Exemplary substitutions include those in Table 1.

TABLE 1

| Original | Exemplary |
|---|---|
| Residue | Substitutions |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr; Ala; Leu |
| Thr | Ser; Ala |
| Trp | Tyr |
| Tyr | Trp; Phe |
| Val | Ile; Leu |

After the substitutions are introduced, the resulting polymerase can be screened for activity by the art worker using assays known to the art worker.

Positions of amino acid residues within a DNA polymerase are indicated by either numbers or number/letter combinations. The numbering starts at the amino terminus residue. The letter is the single letter amino acid code for the amino acid residue at the indicated position in the naturally occurring polymerase from which the mutant is derived. Unless specifically indicated otherwise, an amino acid residue position designation should be construed as referring to the analogous position in all DNA polymerases, even though the single letter amino acid code specifically relates to the amino acid residue at the indicated position in Taq DNA polymerase.

Individual substitution mutations are indicated by the form of a letter/number/letter combination. The letters are the single letter code for amino acid residues. The numbers indicate the amino acid residue position of the mutation site. The numbering system starts at the amino terminus residue. The numbering of the residues in Taq DNA polymerase is as described in U.S. Pat. No. 5,079,352. Amino acid sequence homology between different DNA polymerases permits corresponding positions to be assigned to amino acid residues for DNA polymerases other than Taq. Unless indicated otherwise, a given number refers to position in Taq DNA polymerase. The first letter, i.e., the letter to the left of the number, represents the amino acid residue at the indicated position in the non-mutant polymerase. The second letter represents the amino acid residue at the same position in the mutant polymerase. For example, the term “R660D” indicates that the arginine at position 660 has been replaced by an aspartic acid residue.

Genes encoding DNA polymerases have been isolated and sequenced. This sequence information is available on publicly accessible DNA sequence databases such as GENBANK. A compilation of the amino acid sequences of DNA polymerases from a range of organism can be found in Braithwaite and Ito (1993). This information may be used in designing various embodiments of polymerases of the invention and polynucleotides encoding these polymerases. The publicly available sequence information may also be used to clone genes encoding DNA polymerases through techniques such as genetic library screening with hybridization probes.

### EXAMPLE 1

### Tag G46D, F667Y, S543N Sequencing Performance

The sequencing capabilities of a polymerase of the invention, Taq G46D, F667Y, S543N were investigated. The sequence data from sequencing pGem 3Zf(+) obtained using Taq G46D, F667Y, S543N was compared to data obtained using Taq G46D, F667Y. Comparable data was obtained using both polymerases, indicating that Taq G46D, F667Y, S543N retains its ability to provide accurate sequence data.

Taq G46D, F667Y was used to sequence a template, but Taq G46D, F667Y was not able to proceed past the sequence 5′-GGGGTAGGGGTAGGGGTTGGGG TG-3′ (SEQ ID NO:7) within the template. In contrast Tth 1B21, Tth GK24, rTth FS, Tth Z05, Tth RQ1, and Taq G46D, F667Y, S543N were able to proceed past the sequence that halted Taq G46D, F667Y. (Tth 1B21, Tth GK24, Tth ZO5, Tth RQ1 are strains of Thermus thermophilus; rTth GK24 is a commercially available recombinant Tth available from Roche Molecular Systems). Thus, all of the polymerases from the thermophilus strains were able to read the sequence after SEQ ID NO:7, although some gave weaker signal. Therefore, the behavior of Taq G46D, F667Y, S543N is more like that of the polymerases from strains of Thermus thermophilus than that of Taq G46D, F667Y when the template includes a sequence that stops Taq G46D, F667Y. In PCR reactions Taq G46D, F667Y, S543N also showed a low level of pausing as compared to TaqG46D, F667Y or Taq G46D.

The ability of Taq G46D, F667Y, S543N to sequence pGem3Zf(+) in the presence of varying concentrations of KCl was also assessed. Each polymerase was tested for its ability to sequence pGem3Zf(+) in the presence of 0, 100, and 200 mM KCl. Samples were analyzed on ABI Prism 3100 Genetic Analyzer. Unlike Taq G46D, F667Y, Taq G46D, F667Y, S543N tolerated 100-200 mM KCl. As depicted in Table 2, this was more similar to the results obtained with polymerases derived from thermophilus strains (ZO5 FS, RQ1 FS and TthFS (HB8)). (“FS” refers to the Tabor and Richardson mutation in Taq at position F667Y that reduces bias against the incorporation of dideoxynucleotides (Tabor et al., 1995; U.S. Pat. No. 5,614,365). The designation “FS” in these cases refers to the equivalent position in these Tth strains which may not be exactly at 667 because of differences in the amino acid sequence lengths between Taq and Tth; Tth HB8 is another strain of Thermus thermophilus.)

TABLE 2

| | Total Signal | | |
|---|---|---|---|
| Enzyme | 0 mM KCl | 100 mM KCl | 200 mM KCl |
| AmpliTaq FS | 1791 | 77 | 71 |
| Z05 FS | 3148 | 1575 | 69 |
| RQ1 FS | 3967 | 3107 | 1194 |
| TthFS (HB8) | 3372 | 1514 | 165 |
| Taq G46D, F667Y, S543N | 2590 | 2098 | 686 |

General Methods
Sequencing with BigDye Terminators Version 3.0.

A reaction premix was prepared as described in Table 3 for each reaction:

TABLE 3

| | | |
|---|---|---|
| 5X Buffer¹: | 4 | μL |
| dNTP mix²: | 1 | μL |
| V3 ddA, 8 μM | 0.175 | μL |
| V3 ddC, 30 μM | 0.147 | μL |
| V3 ddG, 4 μM | 0.12 | μL |
| V3 ddU, 40 μM | 26.0 | μL |
| Enzyme (Taq G46D, F667Y, S543N) | 3.32 | μg protein |
| Tth inorganic pyrophosphatase | 5 | units |
| H₂0 to make the final volume 8 μL | | |

¹5X Buffer is 400 mM Tris, pH 9.0, 10 mM MgCl₂ and 0.1% Tween 20.
²The dNTP stock is 4 mM ea dATP, dCTP, dUTP, and 6 mM dITP.

For each sequencing reaction, the premix was combined with plasmid DNA, primer, and water, as follows: 8 μL of reaction premix, 0.25-0.4 μg of plasmid DNA, 3.2 pmoles of primer, and H₂O to make the final volume 20 μL.

Reactions were placed in a thermocycler and reacted following the cycling protocol: 96° C. for 10 seconds, 50° C. for 5 seconds, 60° C. for 4 minutes, for 25 cycles.

The sequencing reactions were then purified using spin columns.

The samples may be treated with SDS, e.g., 2 μL of 2.2% SDS, and heated at 95° C. for 5 minutes prior to the spin column to aid in removal of the unincorporated terminators.

For control reactions with AmpliTaq DNA polymerase FS, a commercial kit containing the BigDye Terminatros V3.0 was used. Samples were analyzed on an ABI Prism 3100 Genetic Analyzer.

PCR Reactions

A Master Mix was prepared for each enzyme tested as follows:

| | |
|---|---|
| 5x Buffer (400 mM Tris pH 9.0, | 20 μl |
| 10 mM MgCl₂, 0.1% Tween 20) | |
| dNTP mix (1.25 mM ea dATP, dCTP, | 16 μl |
| dGTP, dTTP) | |
| Enzyme | 2.5 units or 0.69 μg protein |
| H20 | to make final volume 80 μL |

PCR reactions were set up in 0.2 ml tubes as follows:

| | | |
|---|---|---|
| Master Mix | 80 | μL |
| BigDye-labled Forward primer (Crim F), 10 μM | 1 | μL |
| Unlabeled Reverse primer (Crim 0.5R), 10 μM | 1 | μL |
| Water | 17 | μL |
| Human genomic DNA | 50 | ng |

Samples were analyzed at the 9600 Cycling program: 94° C. 5 sec, 65° C. 1.5 min, hold 4° C.

At the end of the cycling program a 2 μL aliquot was added to 4 μL formamide loading solution for analysis on a 377 gel. 2 μL of this solution was loaded on the gel. The primer peak and PCR peak were off scale.

Reagents for Dye Primer Sequencing

A set of reagent premixes suitable for dye primer sequencing with Taq G46D, S543N, F667Y was prepared as follows:

For each reaction:

A mix: 1 μL 5× buffer (400 mM Tris pH 9.0, 10 mM MgCl₂, 0.1% Tween 20)
   1 μL ddA/dA mix (2 μM ddATP, 500 μM ea dATP, dCTP, c7deazadGTP, dTTP
   1 μL—21 A BigDye Primer (0.4 pmoles/μL)
   0.83 μg Taq G46D, F667Y, S543N in a final volume of 4 μL.
C mix: 1 μL 5× buffer (400 mM Tris pH 9.0, 10 mM MgCl₂, 0.1% Tween 20)
   1 μL ddC/dC mix (2 μM ddCTP, 500 μM ea dATP, dCTP, c7deazadGTP, dTTP
   1 μL—21 C BigDye Primer (0.4 pmoles/μL)
   0.83 μg Taq G46D, F667Y, S543N in a final volume of 4 μL.
G mix: 1 μL 5× buffer (400 mM Tris pH 9.0, 10 mM MgCl₂, 0.1% Tween 20)
   1 μL ddG/dG mix (2 μM ddGTP, 500 μM ea dATP, dCTP, c7deazadGTP, dTTP
   1 μL—21 G BigDye Primer (0.4 pmoles/μL)
   0.83 μg G46D, F667Y, S543N in a final volume of 4 μL.
T mix: 1 μL 5× buffer (400 mM Tris pH 9.0, 10 mM MgCl₂, 0.1% Tween 20)
   1 μL ddT/dT mix (2 μM ddTTP, 500 μM ea DATP, dCTP, c7deazadGTP, dTTP
   1 μL—21 T BigDye Primer (0.4 pmoles/μL)
   0.83 μg Taq G46D, F667Y, S543N in a final volume of 4 μL
      Sequencing reactions for each template were conducted as follows:
A reaction: 1 μL plasmid template at 0.2 μg/μL was combined with 4 μL A mix;
C reaction: 1 μL plasmid template at 0.2 μg/μL was combined with 4 μL C mix;
G reaction: 1 μL plasmid template at 0.2 μg/μL was combined with 4 μL G mix;
T reaction: 1 μL plasmid template at 0.2 μg/μL was combined with 4 μL T mix.

The reactions were thermalcycled in a 9600 (a thermocycler commercially available from Applied Biosystems) using the following program: 96° C. for 10″, 55° C. for 5″, 70° C. for 1 min for 15 cycles followed by 96° C. for 10″, 70° C. for 1 min for 15 cycles.

After the reaction was complete, the products were precipitated with ethanol and loaded on a ABI Prism 3100 Genetic Analyzer for analysis.

### EXAMPLE 2

### Altered Kinetics of Tag G46D, S543N, F667Y

The kinetics of Taq G46D, S543N, F667Y were investigated. It was surprisingly found that Taq G46D, S543N, F667Y displays altered kinetics, e.g., in comparison with the kinetics of Taq G46D, F667Y. The added S543N mutation alters the kinetics of the polymerase by decreasing the polymerase's dissociation rate.

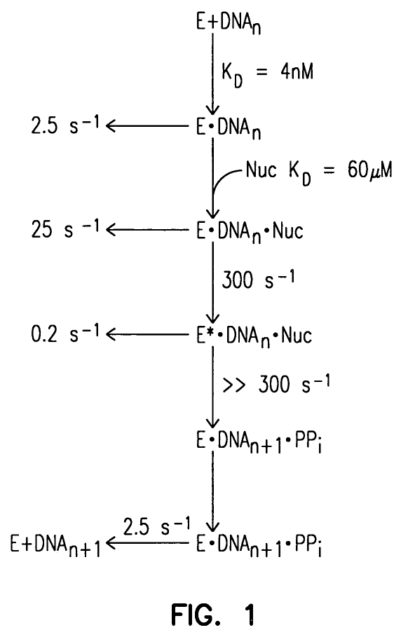

FIG. 1 depicts the two-step nucleotide binding by Taq G46D, F667Y. The diagram shows kinetic steps in the forward polymerization pathway for Taq G46D, F667Y. The polymerase (E) is capable of forming a binary complex with DNA with an equilibrium constant of 4 nM and a dissociation rate of 2.5 s⁻¹. Like other Pol I-type enzymes, Taq G46D, F667Y shows a two-step, induced-fit mechanism for nucleotide (Nuc) discrimination and incorporation. The first step involves the formation of an “open” ternary complex with an equilibrium dissociation constant of 60 μM. Following correct nucleotide binding, the open complex can either rapidly dissociate at about 25 s⁻¹ or form a tighter binding “closed” complex as fast as 300 s⁻¹. The closed complex can either dissociate at a much slower rate of only 0.2 s⁻¹ or undergo a very rapid group transfer reaction to generate a product complex that eventually releases inorganic pyrophosphate (PPi) to begin another round of synthesis under processive conditions (as E·DNAₙ₊₁) or dissociate under “distributive” conditions releasing free enzyme and product (E+DNAₙ₊₁).

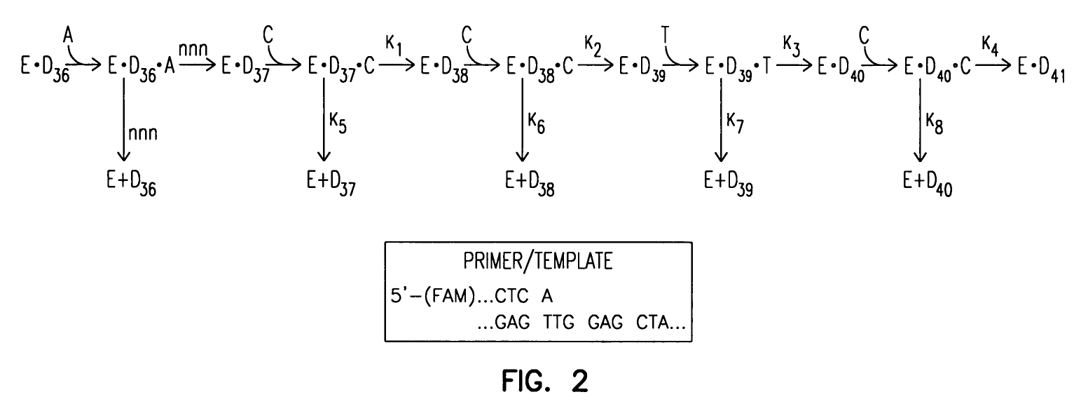

FIG. 2 depicts the principle kinetic steps for processive polymerization for the primer/template shown under conditions where only dATP, dCTP, and dTTP nucleotides were included in the reaction mixture. Polymerization only proceeded as far as the first 5 template positions because dGTP was omitted. It was found that the actual active site concentration determined the magnitudes of the polymerization and off rates for the first step, and only the first step. Therefore, the values shown by “nnn” generated by the curve fitting routine were not included in any of the subsequent calculations for average rates or for processivity and are not included here. The polymerization rates and associated processivity calculations are provided in Tables 4, 5, and 6.

TABLE 4
Processive Polymerization Rates

| | Kinetic Steps | | | | | | | |
|---|---|---|---|---|---|---|---|---|
| Enzyme | 1 | 2 | 3 | 4 | 5 | 6 | 7 | 8 |
| G46D, F667Y | 102 ± 2 | 167 ± 6 | 220 ± 13 | 73 ± 3 | 26 ± 1 | 20 ± 2 | 39 ± 4 | 15 ± 3 |
| G46D, S543N, F667Y | 106 ± 2 | 189 ± 6 | 200 ± 6 | 55 ± 2 | 1 ± 1 | 14 ± 1 | 25 ± 2 | 9 ± 2 |

TABLE 5
Rate Averages

| Enzyme | Average k_{forward} | Average k_{off} |
|---|---|---|
| G46D, F667Y | 141 ± 7 | 25 ± 3 |
| G46D, S543N, F667Y | 138 ± 4 | 12 ± 2 |

* Calculated as the average of the four polymerization rates (k₁ through k₄) and as the average of the four dissociation rates (k₅ through k₈) for each of the mutants as depicted in FIG. 2.

TABLE 6
Processivity Values

| Enzyme | Processivity |
|---|---|
| G46D, F667Y | 6 |
| G46D, S543N, F667Y | 33 |

* Calculated as the average of the ratios of the forward rate divided by the off rate for each round of synthesis taken from Table 1 (Processivity=[k₁/k₅+k₂/k₆+k₃/k₇+k₄/k₈)]/4).

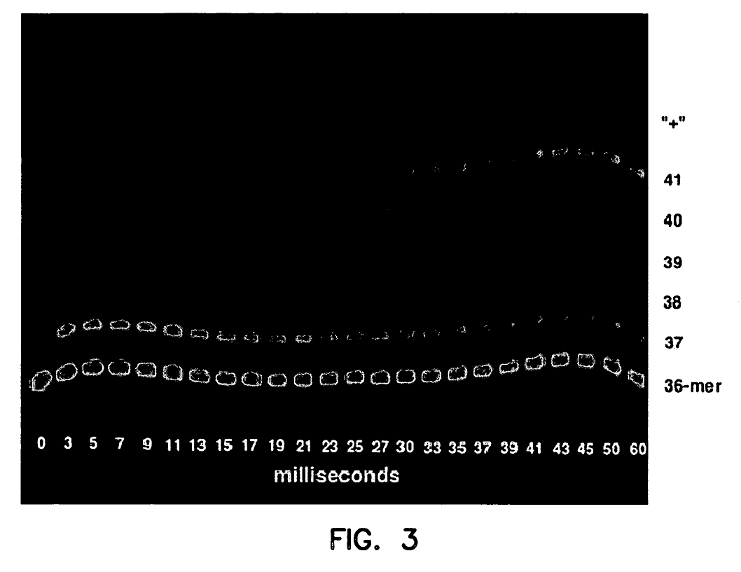

FIG. 3 depicts processive polymerization by Taq G46D, F667Y on 36/45-mer DNA. A preincubated solution containing enyzme (Taq G46D, F667Y 50 nM actual active site concentration or 1 Unit/μL), primer/template DNA (150 nM) plus magnesium chloride (2.4 mM) in buffer (80 mM TRIS-Cl buffer (pH 9.0 at 20° C.) was reacted with dATP, dCTP, and dTTP (400 μM each) in buffer containing 2.4 mM magnesium chloride for the indicated times at 60° C. prior to quenching with 0.5 M EDTA. Samples were resolved on a 16% denaturing polyacrylamide gel using a Model 377 DNA Sequencer and GeneScan software (Applied Biosystems). The bands show the 5′-FAM signal, which represents the flow and accumulation of DNA for each intermediate product through out the time course of the experiment. The numbers on the right axis indicate the template positions and intermediate product sizes. The “+” designates bands representing probable misincorporation occurring at the 42nd-template position since the template base at position 42 was C and no dGTP was present in the reaction mixture. The bands below the 36-mer primer correspond to a “capped” by-product generated during the chemical synthesis of the primer which failed to be removed by FPLC-reversed-phase purification of the fragments. This DNA did not participate in the reaction and its mass contribution to the overall DNA concentration was corrected in the calculations.

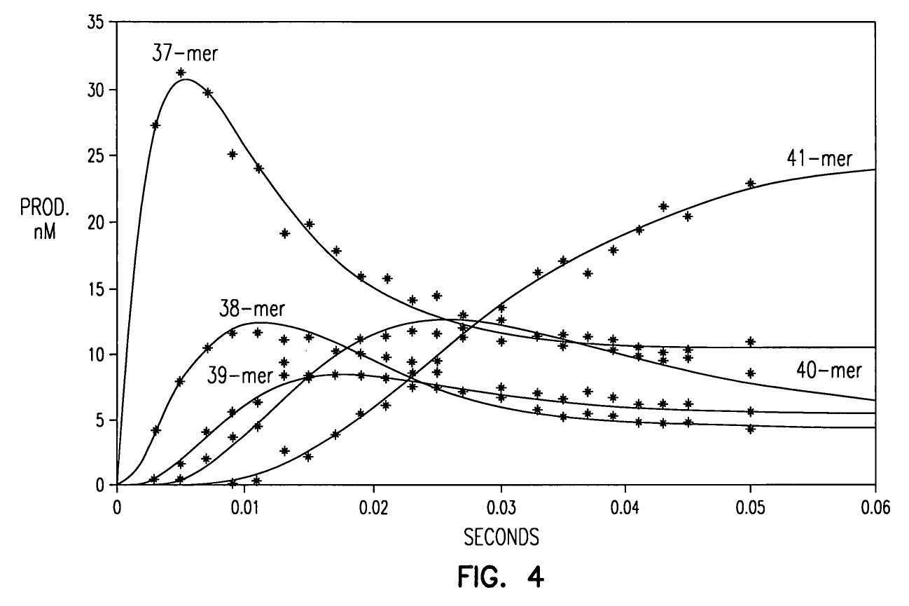

FIG. 4 depicts processive polymerization by Taq G46D, F667Y on 36/45-mer DNA. The fluorescent signal in each of the bands shown in FIG. 3 was converted to nM of DNA by normalization (see Brandis et al., 1996) and plotted versus time as shown. The solid lines represent the best fits obtained from computer simulation using a mechanism of a series of five nucleotide incorporations and enzyme dissociations as depicted in FIG. 2. If Taq G46D, F667Y had dissociated from the primer/template with a rate of only 2.5 s⁻¹ as predicted by the binary dissociation rate shown in FIG. 1, then each of the intermediate product lines should have returned to baseline during the time course of this experiment. These lines did not return to baseline, indicating that a significant portion of the polymerization complex dissociated after each round of incorporation.

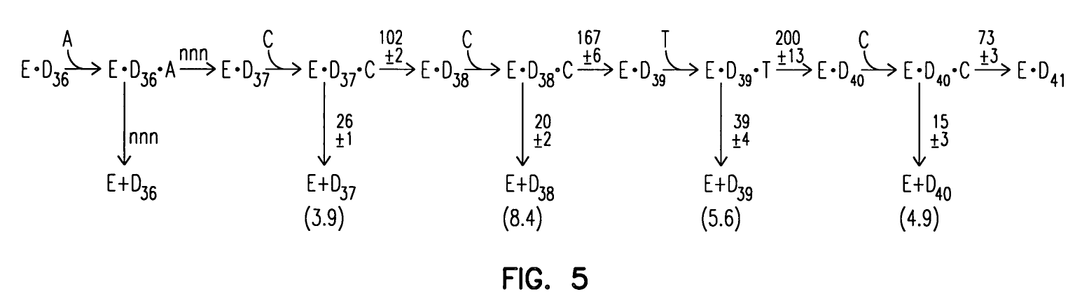

FIG. 5 depicts the polymerization and dissociation rates (each±one standard deviation) for Taq G46D, F667Y as determined by non-linear curve fitting to the data points shown in FIG. 4. The average polymerization rate for Taq G46D, F667Y was 141±7 s⁻¹ and the average dissociation rate was 25±3 s⁻¹. The numbers in parentheses represent the ratios of the forward rate divided by the off rate for each round of polymerization. The calculated processivity value determined as the average of these ratios was only 6. The value determined using this pre-steady-state approach for Taq G46D, F667Y was much lower than the published value of >60 for Taq F667Y measured using a gel-based assay by Innis et al., 1988.

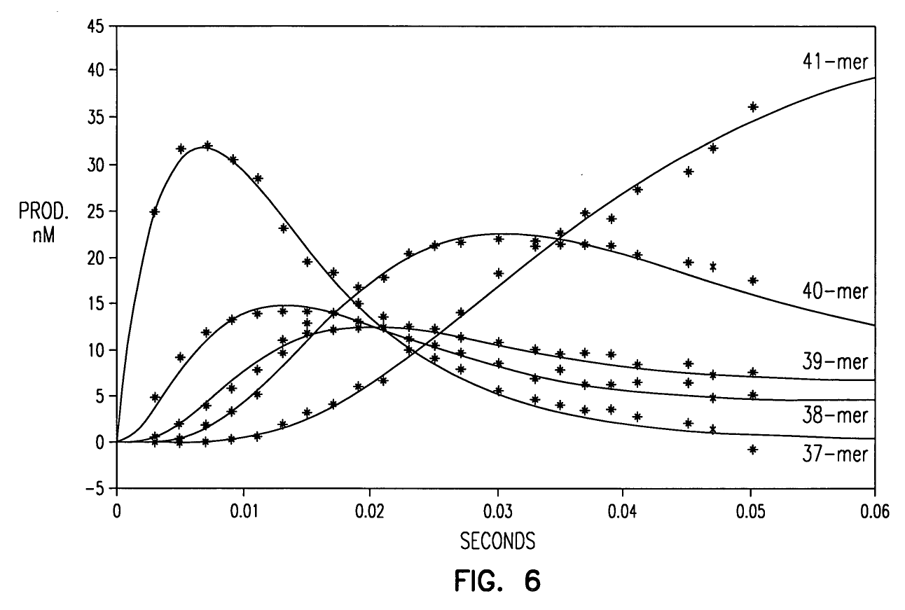

FIG. 6 depicts processive polymerization by Taq G46D, S543N, F667Y on 36/45-mer DNA. Experimental conditions and determinations were the same as those described in FIGS. 3 and 4. These lines also represent the best fits to the data points. Unlike the case for Taq G46D, F667Y, some of these lines nearly return to baseline, indicating slower dissociation rates during each round of polymerization.

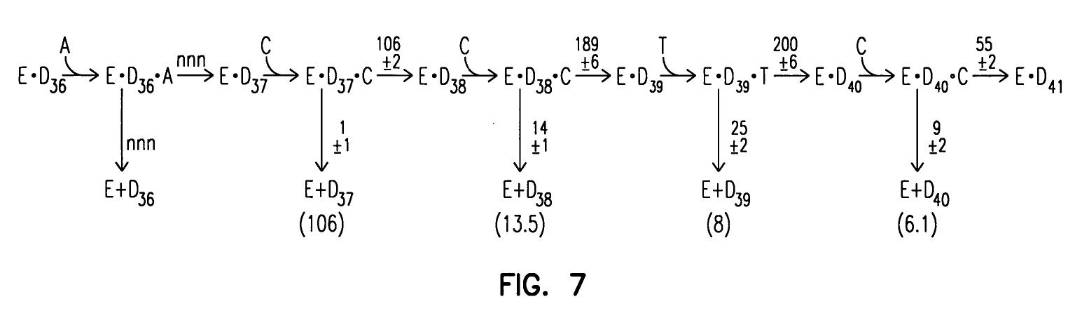

FIG. 7 depicts a processive polymerization pathway for Taq G46D, S543N, F667Y and shows the rate measurements for the triple mutant. Polymerization rates were not significantly different than those measured for Taq G46D, F667Y, but the dissociation rates were slower, especially for incorporation of the first C in the second round of polymerization. The average polymerization rate for Taq G46D, S543N, F667Y was 138±4 s⁻¹ and the average dissociation rate was 12±2 s⁻¹. The calculated processivity value determined as the average of the ratios shown in the parentheses was 33 or about 6× higher than Taq G46D, F667Y.

All publications, patents and patent applications cited herein are herein incorporated by reference.

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

Documents Cited

U.S. Pat. No. 5,079,352.
U.S. Pat. No. 5,405,774.
U.S. Pat. No. 5,455,170.
U.S. Pat. No. 5,466,591.
U.S. Pat. No. 5,614,365
U.S. Pat. No. 5,795,762.
U.S. Pat. No. 6,265,193.
Abramson, in Innis et al., PCR Applications: Protocols for Functional Genomics, Academic Press, 33-47 (1999).
Braithwaite and Ito, Nucl. Acids Res, 21(4), 787-802 (1993).
Brandis et al., Biochemistry, 35(7), 2189-200 (1996).
Innis et al., PNAS, 85, 9436 (1988).
Joyce and Steitz, Ann. Rev. Biochem. 63:777-822 (1994).
Tabor et al., PNAS, 92, 6339-6343 (1995).
Kalman et al., Genome Science and Technology, 1, 42, (1995).
Kornberg, DNA Replication, Second Edition, W. H. Freeman (1989).
Ignatov et al., FEBS Letters, 425, 249-250 (1998).
Ignatov et al., FEBS Letters, 448, 145-148 (1999).
Molecular Cloning: A Laboratory Manual (Sambrook et al, 3rd Ed., Cold Spring Harbor Laboratory Press, (2001).
Xu et al., J. Mol. Biol., 268(2), 284-302 (1997).

What is claimed is:

1. A mutant DNA polymerase comprising an Asn residue at amino acid 543 and a 5′-3′ exonuclease activity reducing mutation, wherein the positions of amino acids of the mutant DNA polymerase are defined with respect to Taq DNA polymerase.

2. The mutant polymerase of claim 1, wherein the 5′-3′ exonuclease activity reducing mutation is an N-terminal deletion.

3. The mutant polymerase of claim 1, wherein the 5′-3′ exonuclease activity reducing mutation is an Asp residue at amino acid 46.

4. The mutant polymerase of claim 1, further comprising a Tyr residue at amino acid 667.

5. The mutant polymerase of claim 1 that is a thermostable DNA polymerase.

6. The mutant polymerase of claim 1 that is a mutant Taq DNA polymerase.

7. The mutant polymerase of claim 1 that is a thermostable mutant Taq DNA polymerase.

8. The mutant polymerase of claim 1 that comprises SEQ ID NO:3 or SEQ ID NO:5.

9. A polynucleotide comprising a sequence encoding the polymerase of claim 1.

10. A polynucleotide comprising a sequence encoding the polymerase of claim 4.

11. A polynucleotide comprising a sequence encoding the polymerase of claim 8.

12. The polynucleotide of claim 11 that comprises SEQ ID NO:4 or SEQ ID NO:6.

13. A vector comprising the polynucleotide of claim 9.

14. A vector comprising the polynucleotide of claim 10.

15. A vector comprising the polynucleotide of claim 11.

16. The vector of claim 13, further comprising a promoter operably linked to the polynucleotide.

17. The vector of claim 14, further comprising a promoter operably linked to the polynucleotide.

18. The vector of claim 15, further comprising a promoter operably linked to the polynucleotide.

19. A cell comprising the DNA polymerase of claim 1.

20. A cell comprising the polynucleotide of claim 9.

21. A cell comprising the vector of claim 13.

22. A method for synthesizing a polynucleotide in a reaction, comprising contacting the mutant polymerase of claim 1 with a primed template and nucleotides.

23. The method of claim 22, wherein the reaction is a chain termination sequencing reaction.

24. The method of claim 22, wherein the reaction is a polymerase chain reaction.

25. The method of claim 22, wherein the nucleotides comprise labeled nucleotides.

26. The method of claim 25, wherein the labeled nucleotides are fluorescently labeled nucleotides.

27. A kit comprising packaging material and the mutant polymerase of claim 1.

28. The kit of claim 27, further comprising labeled nucleotides.

29. The kit of claim 28, wherein the labeled nucleotides are fluorescently labeled nucleotides.

30. The kit of claim 27, further comprising unlabeled nucleotides.

31. The kit of claim 27, further comprising at least one primer.
